(19) United States
(12) Patent Application Publication
(10) Pub. No.: US 2008/0242722 A1
(43) Pub. Date: Oct. 2, 2008

(54) Combination Therapy for Treating Cyclooxygenase-2 Mediated Diseases or Conditions in Patients at Risk of Thrombotic Cardiovascular Events

(76) Inventors: Claude Dufresne, Dollard-des-Ormeaux (CA); Carl Berthelette, Ste-Dorothee Laval (CA); Lianhai Li, Montreal (CA); Daniel Guay, Vaudreuil-Dorion (CA); Michel Gallant, Kirkland (CA); Patrick Lacombe, Montreal (CA); Renee Aspiotis, Kirkland (CA); Zhaoyin Wang, Kirkland (CA); Claudio Sturino, L'ile-Bizard (CA)

Correspondence Address: MERCK AND CO., INC, P O BOX 2000, RAHWAY, NJ, 07065-0907, US

(21) Appl. No.: 10/586,407

(22) PCT Filed: Jan. 25, 2005
(86) PCT No.: PCT/CA05/00082

§ 371 Date: Jul. 18, 2006

Related U.S. Application Data
(60) Provisional application No. 60/539,912, filed on Jan. 27, 2004.

(51) Int. Cl. A61K 31/365; A61K 31/22; A61K 31/045; A61P 19/02; A61K 31/04

(52) U.S. Cl. 514/473; 514/509; 514/727; 514/742

(57) ABSTRACT

The invention is directed to a method for treating a cyclooxygenase-2 mediated disease or condition in a mammalian patient at risk of a thrombotic cardiovascular event, wherein the patient is on aspirin therapy to reduce the risk of the thrombotic cardiovascular event,

comprising orally concomitantly or sequentially administering to the patient a cyclooxygenase-2 selective inhibitor in an amount effective to treat the cyclooxygenase-2 mediate disease or condition, and
a nitric oxide donating compound in accordance with Formula I

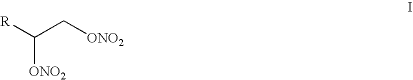

or a pharmaceutically acceptable salt thereof, wherein the nitric oxide donating compound is administered in an amount effective to reduce the gastrointestinal toxicity caused by the combination of the cyclooxygenase-2 selective inhibitor and aspirin. Pharmaceutical compositions are also encompassed.

### BACKGROUND OF THE INVENTION

Selective inhibitors of cyclooxygenase-2 are a sub-class of the class of drugs known as non-steroidal antiinflammatory drugs (NSAIDs). The NSAIDs are active in reducing the prostaglandin-induced pain and swelling associated with the inflammation process but are also active in affecting other prostaglandin-regulated processes not associated with the inflammation process. Thus, use of high doses of most common NSAIDs can produce severe side effects, including life threatening ulcers, that limit their therapeutic potential. An alternative to NSAIDs is the use of corticosteroids, which have even more drastic side effects, especially when long term therapy is involved.

Previous NSAIDs have been found to prevent the production of prostaglandin by inhibiting enzymes in the human arachidonic acid/prostaglandin pathway including the enzyme cyclooxygenase (COX). The discovery that there are two isoforms of the COX enzyme, the first, COX-1, being involved with physiological functions and the second, COX-2, being induced in inflamed tissue, has given rise to a new approach. While conventional NSAIDs block both forms of the enzyme, the identification of the inducible COX-2 enzyme associated with inflammation has provided a viable target of inhibition which more effectively reduces inflammation and produces fewer and less drastic side effects. Many compounds which have activity as COX-2 inhibitors have been identified, including rofecoxib (VIOXX®), etoricoxib (ARCOXIA™), celecoxib (CELEBREX®) and valdecoxib (BEXTRA™), and much research continues in this area.

Many patients with a chronic cyclooxygenase-2 mediated disease or condition are elderly and thus are at increased risk for thrombotic cardiovascular events, such as stroke, myocardial ischemia, myocardial infarction, angina pectoris, transient ischemic attack (TIA; amaurosis fugax), reversible ischemic neurologic deficits, and any similar thrombotic event in any vascular bed (splancnic, renal, aortic, peripheral, etc.). Moreover, there is evidence that patients with chronic inflammatory conditions, such as rheumatoid arthritis and systemic lupus erythematosis are at increased risk for thrombotic cardiovascular events. It is desirable that such patients receive appropriate therapy to reduce their risk of such events, such as low-dose aspirin therapy. However, it has been reported that the co-administration of aspirin and a selective COX-2 inhibitor in a rat model resulted in substantially more severe gastric injury than is produced with either agent alone. See Fiorucci et al., Gastroenterology, vol. 123, pp. 1598-1606, 2002. Thus, the major advantage that COX-2 selective inhibitors have over NSAIDS may be offset by the concomitant use of aspirin.

The present invention provides for a combination of a specific class of 1,2-dinitrate compounds that when co-administered with a selective cyclooxygenase-2 and aspirin provides for a surprisingly-improved gastrointestinal safety profile relative to the combination of the cyclooxygenase-2 selective inhibitor and aspirin without the nitric oxide releasing compound. Thus, the invention provides efficacy in treating cyclooxygenase-2 mediated diseases or conditions, effectively inhibits platelets thus reducing the risk of thrombotic cardiovascular events and potentially renal side effects and at the same time reduces the risk of GI ulceration or bleeding relative to the co-administration of a COX-2 inhibitor and low-dose aspirin.

### SUMMARY OF THE INVENTION

The invention is directed to a method for treating a cyclooxygenase-2 mediated disease or condition in a mammalian patient at risk of a thrombotic cardiovascular event, wherein the patient is on aspirin therapy to reduce the risk of the thrombotic cardiovascular event,

comprising orally concomitantly or sequentially administering to the patient a cyclooxygenase-2 selective inhibitor in an amount effective to treat the cyclooxygenase-2 mediate disease or condition, and
a nitric oxide donating compound in accordance with Formula I

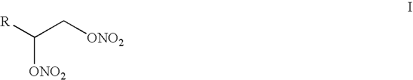

or a pharmaceutically acceptable salt thereof, wherein the nitric oxide donating compound is administered in an amount effective to reduce the gastrointestinal toxicity caused by the combination of the cyclooxygenase-2 selective inhibitor and aspirin. Pharmaceutical compositions are also encompassed.

### DETAILED DESCRIPTION OF THE INVENTION

The invention encompasses a method for treating a cyclooxygenase-2 mediated disease or condition in a human patient at risk of a thrombotic cardiovascular event, wherein the patient is on aspirin therapy to reduce the risk of the thrombotic cardiovascular event, comprising orally concomitantly or sequentially administering to the patient a cyclooxygenase-2 selective inhibitor in an amount effective to treat the cyclooxygenase-2 mediate disease or condition, and

a nitric oxide donating compound in accordance with Formula I

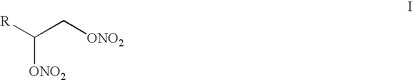

or a pharmaceutically acceptable salt thereof, wherein R is selected from the group consisting of:

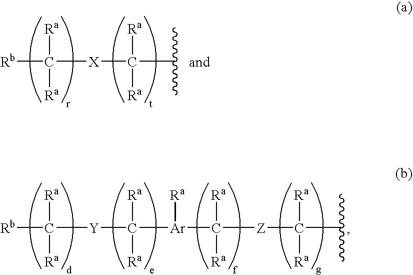

wherein:

r and t are independently 0 to 10,

d, e, f and g are independently 0 to 10;

Ar is selected from the group consisting of: phenyl, naphthyl, biphenyl and HET,

X, Y and Z are independently selected from the group consisting of: a bond, —O—, —S(O)ₖ—, —O—C(O)—, —C(O)—O— and —O—C(O)—O—, with the proviso that when X is other than a bond then r is not 0, and with the proviso that when Y is other than a bond then d is not 0, and wherein k is 0, 1 or 2,

each R^{a} and R^{b} are independently selected from the group consisting of:
(1) hydrogen,
(2) halo,
(3) C₁₋₄alkoxy,
(4) C₁₋₄alkylthio,
(5) OH,
(6) CN,
(7) CO₂R^{c} and
(8) —C₀₋₆—ONO₂,

and two R^{a} groups on the same carbon atom may be joined together to form a carbonyl group;

R^{c} is selected from the group consisting of: hydrogen and C₁₋₆alkyl; and

HET is selected from the group consisting of: benzimidazolyl, benzofuranyl, benzopyrazolyl, benzotriazolyl, benzothiophenyl, benzoxazolyl, carbazolyl, carbolinyl, cinnolinyl, furanyl, imidazolyl, indolinyl, indolyl, indolazinyl, indazolyl, isobenzofuranyl, isoindolyl, isoquinolyl, isothiazolyl, isoxazolyl, naphthyridinyl, oxadiazolyl, oxazolyl, pyrazinyl, pyrazolyl, pyridopyridinyl, pyridazinyl, pyridyl, pyrimidyl, pyrrolyl, quinazolinyl, quinolyl, quinoxalinyl, thiadiazolyl, thiazolyl, thienyl, triazolyl, azetidinyl, 1,4-dioxanyl, hexahydroazepinyl, piperazinyl, piperidinyl, pyrrolidinyl, morpholinyl, thiomorpholinyl, dihydrobenzimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, and tetrahydrothienyl,
wherein the nitric oxide donating compound is administered in an amount effective to reduce the gastrointestinal toxicity caused by the combination of the cyclooxygenase-2 selective inhibitor and aspirin.

An embodiment of the invention encompasses the above method wherein the compound of Formula I and the cyclooxygenase-2 selective inhibitor are administered concomitantly.

Another embodiment of the invention encompasses the above method wherein the compound of Formula I and the cyclooxygenase-2 selective inhibitor are administered sequentially.

Another embodiment of the invention encompasses the above method wherein the cyclooxygenase-2 selective inhibitor is selected from the group consisting of: rofecoxib, etoricoxib, celecoxib, valdecoxib, lumiracoxib, LAS34475 and GW406381. Within this embodiment, the invention encompasses the above method wherein the cyclooxygenase-2 selective inhibitor is rofecoxib.

Another embodiment of the invention encompasses the above method wherein R is

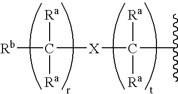

wherein X is a bond, each R^{a} is hydrogen and R^{b} is OH.

Another embodiment of the invention encompasses the above method wherein R is

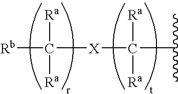

wherein X is a —O—C(O)— or —C(O)—O—, each R^{a} is hydrogen and R^{b} is hydrogen and both r and t are greater than 0.

Another embodiment of the invention encompasses the above method wherein R is

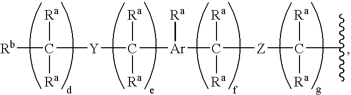

wherein Ar is phenyl,
f and e are both 0 and

Z and Y are independently —S(O)₂—, —O—C(O)— or —C(O)—O—.

Another embodiment of the invention encompasses the above method wherein the nitric oxide donating compound in accordance with Formula I is selected from the group consisting of:

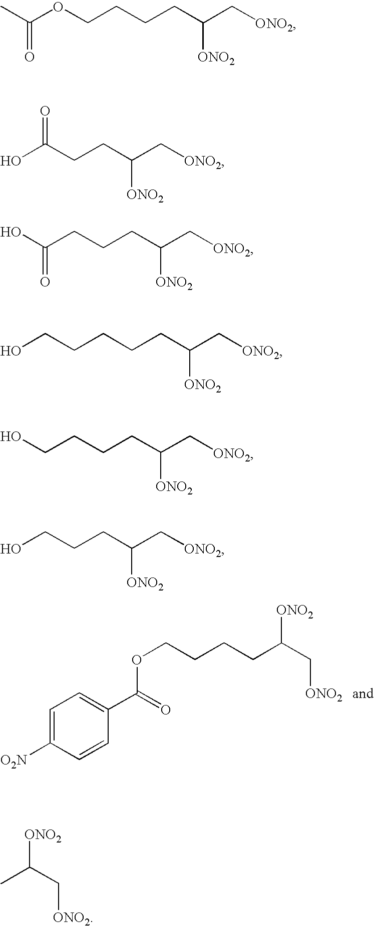

Another embodiment of the invention encompasses the above method wherein R is selected from

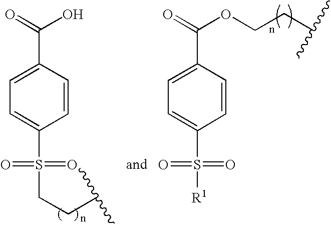

wherein R¹ is C₁₋₆alkyl and n is 1 to 8.

Another embodiment of the invention encompasses the above method wherein the thrombotic cardiovascular event is selected from the group consisting of: thrombotic or thromboembolic stroke, myocardial ischemia, myocardial infarction, angina pectoris, transient ischemic attack and reversible ischemic neurologic deficits.

Another embodiment of the invention encompasses the above method wherein the cyclooxygenase-2 mediated disease or condition is selected from the group consisting of: osteoarthritis, rheumatoid arthritis, chronic and acute pain, primary dysmenorrhea, gout, ankylosing spondylitis and bursitis.

Another embodiment of the invention encompasses a pharmaceutical composition comprising a nitric oxide donating compound in accordance with Formula I

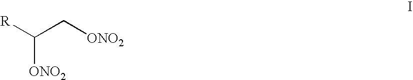

or a pharmaceutically acceptable salt thereof, wherein R is selected from the group consisting of:

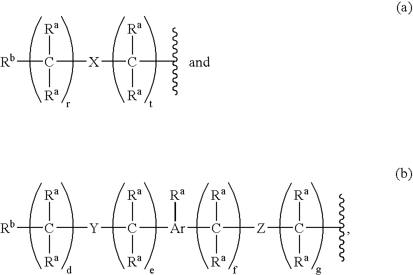

wherein:

r and t are independently 0 to 10,

d, e, f and g are independently 0 to 10;

Ar is selected from the group consisting of: phenyl, naphthyl, biphenyl and HET,

X, Y and Z are independently selected from the group consisting of: a bond, —O—, —S(O)ₖ—, —O—C(O)—, —C(O)—O— and —O—C(O)—O—, with the proviso that when X is other than a bond then r is not 0, and with the proviso that when Y is other than a bond then d is not 0, and wherein k is 0, 1 or 2,

each R^{a} and R^{b} are independently selected from the group consisting of:
(1) hydrogen,
(2) halo,
(3) C₁₋₄alkoxy,
(4) C₁₋₄alkylthio,
(5) OH,
(6) CN,
(7) CO₂R^{c},
(8) —C₀₋₆—ONO₂,

R^{c} is selected from the group consisting of: hydrogen and C₁₋₆alkyl; and

HET is selected from the group consisting of: benzimidazolyl, benzofuranyl, benzopyrazolyl, benzotriazolyl, benzothiophenyl, benzoxazolyl, carbazolyl, carbolinyl, cinnolinyl, furanyl, imidazolyl, indolinyl, indolyl, indolazinyl, indazolyl, isobenzofuranyl, isoindolyl, isoquinolyl, isothiazolyl, isoxazolyl, naphthyridinyl, oxadiazolyl, oxazolyl, pyrazinyl, pyrazolyl, pyridopyridinyl, pyridazinyl, pyridyl, pyrimidyl, pyrrolyl, quinazolinyl, quinolyl, quinoxalinyl, thiadiazolyl, thiazolyl, thienyl, triazolyl, azetidinyl, 1,4-dioxanyl, hexahydroazepinyl, piperazinyl, piperidinyl, pyrrolidinyl, morpholinyl, thiomorpholinyl, dihydrobenzimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, and tetrahydrothienyl,
and a cyclooxygenase-2 selective inhibitor in combination with a pharmaceutically acceptable carrier.

Another embodiment of the invention encompasses the above pharmaceutical composition wherein the cyclooxygenase-2 selective inhibitor is selected from the group consisting of: rofecoxib, etoricoxib, celecoxib, valdecoxib, lumiracoxib, LAS34475 and GW406381. Within this embodiment, the invention encompasses the above pharmaceutical composition wherein the cyclooxygenase-2 selective inhibitor is rofecoxib. Also within this embodiment, the invention encompasses the above pharmaceutical composition wherein the cyclooxygenase-2 selective inhibitor is rofecoxib which is present in amount selected from the group consisting of 12.5 mg, 25 mg and 50 mg.

The invention also encompasses a pharmaceutical composition comprising rofecoxib present in an amount selected from the group consisting of 12.5 mg, 25 mg and 50 mg and an NO-donating compound having the following formula:

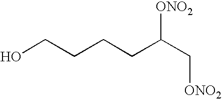

in combination with a pharmaceutically acceptable carrier.

Another embodiment of the invention encompasses a pharmaceutical composition comprising a nitric oxide donating compound in accordance with Formula I

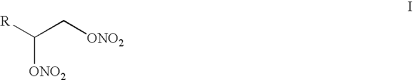

or a pharmaceutically acceptable salt thereof, wherein R is

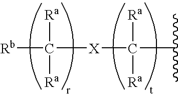

X is a bond, each R^{a} is hydrogen and R^{b} is OH,
and a cyclooxygenase-2 selective inhibitor in combination with a pharmaceutically acceptable carrier.

Another embodiment of the invention encompasses a pharmaceutical composition comprising a nitric oxide donating compound in accordance with Formula I

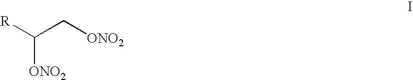

or a pharmaceutically acceptable salt thereof, wherein R is

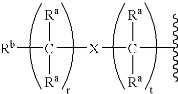

wherein X is a —O—C(O)— or —(O)—O—, each R^{a} is hydrogen and R^{b} is hydrogen and both r and t are greater than 0,
and a cyclooxygenase-2 selective inhibitor in combination with a pharmaceutically acceptable carrier.

Another embodiment of the invention encompasses a pharmaceutical composition comprising a nitric oxide donating compound in accordance with Formula I

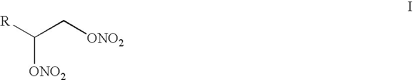

or a pharmaceutically acceptable salt thereof, wherein R is

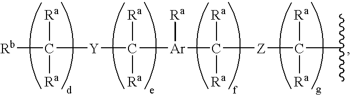

wherein Ar is phenyl,
f and e are both 0 and
Z and Y are independently S(O)₂, —O—C(O)— or —C(O)—O—,
and a cyclooxygenase-2 selective inhibitor in combination with a pharmaceutically acceptable carrier.

Another embodiment of the invention encompasses a pharmaceutical composition comprising a nitric oxide donating compound selected from the group consisting of:

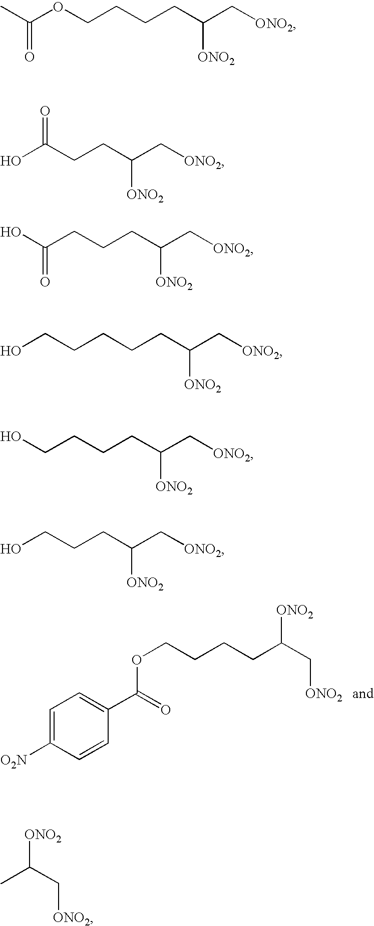

and a cyclooxygenase-2 selective inhibitor in combination with a pharmaceutically acceptable carrier.

Another embodiment of the invention encompasses a pharmaceutical composition comprising a nitric oxide donating compound in accordance with Formula I

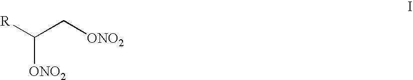

or a pharmaceutically acceptable salt thereof, wherein R is

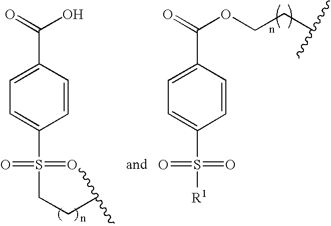

wherein R¹ is C₁₋₆alkyl and n is 1 to 8, and a cyclooxygenase-2 selective inhibitor in combination with a pharmaceutically acceptable carrier.

Another embodiment of the invention encompasses a method for treating a cyclooxygenase-2 mediated disease or condition comprising administering the pharmaceutical compositions described above. Within this embodiment is encompassed the method wherein the cyclooxygenase-2 mediated disease or condition is selected from the group consisting of: osteoarthritis, rheumatoid arthritis, chronic and acute pain, primary dysmenorrhea, gout, ankylosing spondylitis and bursitis. Also within this embodiment is encompassed the method wherein the patient is at risk of a thrombotic cardiovascular event and wherein the patient is on aspirin therapy to reduce the risk of the thrombotic cardiovascular event. The invention also encompasses this method wherein the cardiovascular event is selected from the group consisting of: thrombotic or thromboembolic stroke, myocardial ischemia, myocardial infarction, angina pectoris, transient ischemic attack and reversible ischemic neurologic deficits.

In another embodiment of the invention, the patient to which the pharmaceutical composition is being administered has had ischemic stroke or transient ischemia of the brain due to fibrin platelet emboli and said patient is on aspirin therapy of about 50 to about 325 mg once daily. In another embodiment, the patient had a previous myocardial infarction or has unstable angina pectoris and said patient is on aspirin therapy of about 75 to about 325 mg once daily. In another embodiment, the patient has chronic stable angina pectoris and said patient is on aspirin therapy of about 75 to about 325 mg once daily.

The following abbreviations have the indicated meanings:

AD-mix-alpha=reagent for asymmetric Sharpless dihydroxylation (commercially available)

AD-mix-beta=reagent for asymmetric Sharpless dihydroxylation (commercially available)

DMAP=4-(dimethylamino)pyridine

EA=ethyl acetate

Hex=hexane

RT=room temperature

TBF=tetrahydrofuran

The term “treating a chronic cylcooxygenase-2 mediated disease or condition” means treating or preventing any acute or chronic disease or condition that is treated or prevented by inhibiting the cyclooxygenase-2 enzyme. The term includes the relief of pain, fever and inflammation of a variety of conditions including rheumatic fever, symptoms associated with influenza or other viral infections, common cold, low back and neck pain, dysmenorrhea, headache, migraine (acute and prophylactic treatment), toothache, sprains and strains, myositis, neuralgia, synovitis, arthritis, including rheumatoid arthritis, degenerative joint diseases (osteoarthritis), gout and ankylosing spondylitis, acute, subacute and chronic musculoskeletal pain syndromes such as bursitis, burns, injuries, and pain following surgical and dental procedures as well as the preemptive treatment of surgical pain. In addition, the term includes the inhibition cellular neoplastic transformations and metastic tumor growth and hence the treatment of cancer. The term also includes the treatment of endometriosis and Parkinson's disease as well as the treatment of cyclooxygenase-mediated proliferative disorders such as may occur in diabetic retinopathy and tumor angiogenesis. The term “treating” encompasses not only treating a patient to relieve the patient of the signs and symptoms of the disease or condition but also prophylactically treating an asymptomatic patient to prevent the onset or progression of the disease or condition.

A “thrombotic cardiovascular event” is defined as any sudden event of a type known to be caused by platelet aggregation, thrombosis, and subsequent ischemic clinical events, including thrombotic or thromboembolic stroke, myocardial ischemia, myocardial infarction, angina pectoris, transient ischemic attack (TIA; amaurosis fugax), reversible ischemic neurologic deficits, and any similar thrombotic event in any vascular bed (splancnic, renal, aortic, peripheral, etc.).

A “patient at risk of a thrombotic cardiovascular event” can readily be diagnosed by one having ordinary skill in the art. For example, such a patient includes those who have had ischemic stroke or transient ischemia of the brain due to fibrin platelet emboli, those with a previous myocardial infarction or unstable angina pectoris, and those with chronic stable angina pectoris. Risk factors for a thrombotic cardiovascular event include hypertension, hypercholesterolemia, diabetes mellitus, chronic renal impairment, smoking, and any prior personal or family history of such an event. A patient with one or more of the aforementioned risk factors is included within the scope of the present invention. The patient at risk of a thrombotic cardiovascular event according to the invention would be on aspirin therapy to reduce the risk of the cardiovascular event. The term “aspirin therapy to reduce the risk of the cardiovascular event” is well understood in the art as low-dose aspirin is indicated for these conditions. Administration of the drug combination to the patient includes both self-administration and administration to the patient by another person.

The term “amounts that are effective to treat” is intended to mean that amount of a drug or pharmaceutical agent that will elicit the biological or medical response of a tissue, a system, animal or human that is being sought by a researcher, veterinarian, medical doctor or other clinician. The term also encompasses the amount of a pharmaceutical drug that will prevent or reduce the risk of occurrence of the biological or medical event that is sought to be prevented in a tissue, a system, animal or human by a researcher, veterinarian, medical doctor or other clinician. The inhibitor of cyclooxygenase-2 may be administered at a dosage level up to conventional dosage levels for NSAIDs. Suitable dosage levels of the compound of Formula I used in the present invention are described below. The compound may be administered on a regimen of once or twice per day.

For vascular indications, aspirin is typically administered at a dose of about 30 mg to about 1 g once daily, preferably at a dose of about 80 mg to about 650 mg.

The term “amount effective to reduce the gastrointestinal toxicity caused by the combination of the cyclooxygenase-2 selective inhibitor and aspirin” means the amount of the compound of Formula I that will deliver sufficient nitric oxide to reduce the gastrointestinal toxicity caused by the combination of the cyclooxygenase-2 selective inhibitor and aspirin. Suitable dosage levels are discernable by those having ordinary skill in the art, but typically suitable levels will be in the range of about 1 to about 1000 mg.

The term “concomitantly administering” means administering the agents substantially concurrently. The term “concomitantly administering” encompasses not only administering the two agents in a single pharmaceutical dosage form but also the administration of each active agent in its own separate pharmaceutical dosage formulation. Where separate dosage formulations are used, the agents can be administered at essentially the same time, i.e., concurrently.

The term “sequentially administering” means administering the agents at separately staggered times. Thus, agents can be sequentially administered such that the beneficial pharmaceutical effect of NO-donating compound of Formula I and the COX-2 inhibitor are realized by the patient at substantially the same time. Thus, for example, if a COX-2 selective inhibitor and NO donating compound of Formula I are both administered on a once a day basis, the interval of separation between sequential administration of the two agents can be up to twelve hours apart.

The pharmaceutical compositions of the present invention comprise a cycloxygenase-2 selective inhibitor and a compound of Formula I as active ingredients, or pharmaceutically acceptable salts thereof, and may also contain a pharmaceutically acceptable carrier and optionally other therapeutic ingredients. The term “pharmaceutically acceptable salts” include salts prepared from bases that result in non-toxic pharmaceutically acceptable salts, including inorganic bases and organic bases. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

When the compounds of the present invention is basic, salts may be prepared from acids that result in pharmaceutically acceptable salts, including inorganic and organic acids. Such acids include acetic, adipic, aspartic, 1,5-naphthalenedisulfonic, benzenesulfonic, benzoic, camphorsulfonic, citric, 1,2-ethanedisulfonic, ethanesulfonic, ethylenediaminetetraacetic, fumaric, glucoheptonic, gluconic, glutamic, hydriodic, hydrobromic, hydrochloric, isethionic, lactic, maleic, malic, mandelic, methanesulfonic, mucic, 2-naphthalenesulfonic, nitric, oxalic, pamoic, pantothenic, phosphoric, pivalic, propionic, salicylic, stearic, succinic, sulfuric, tartaric, p-toluenesulfonic acid, undecanoic, 10-undecenoic, and the like.

The term “cyclooxygenase-2 selective inhibitors” means compounds that are inhibitors of cyclooxygenase-2 enzyme and are thereby useful in the treatment of cyclooxygenase-2 mediated diseases as enumerated above. This activity is illustrated by their ability to selectively inhibit cyclooxygenase-2 over cyclooxygenase-1. Accordingly, in one assay, the ability of the compounds of this invention to treat cyclooxygenase mediated diseases can be demonstrated by measuring the amount of prostaglandin E₂ (PGE₂) synthesized in the presence of arachidonic acid, cyclooxygenase-1 or cyclooxygenase-2 and a compound of Formula I. The IC₅₀ values represent the concentration of inhibitor required to return PGE₂ synthesis to 50% of that obtained as compared to the uninhibited control. The terms “cyclooxygenase-2 selective inhibitor” includes compounds in any form, including salts, hydrates and polymorphs.

The nitric oxide released by the compound of Formula I is believed to contribute to an improved gastrointestinal and potentially renal safety profile. Thus, the present invention provides treatment of chronic cyclooxygenase-2 mediated diseases or conditions, effectively reduces the risk of thrombotic cardiovascular events and potentially renal side effects and at the same time reduces the risk of GI ulceration or bleeding. Thus, patients with hypertension and cardiovascular disease, as well as potentially patients with renal insufficiency, would actively benefit from being administered the combination of the present invention over NSAIDs and cyclooxygenase-2 selective inhibitors currently available.

The combination of this invention will be useful for the relief of pain, fever and inflammation of a variety of conditions including rheumatic fever, symptoms associated with influenza or other viral infections, common cold, low back and neck pain, dysmenorrhea, headache, migraine (acute and prophylactics treatment), toothache, sprains and strains, myositis, neuralgia, synovitis, arthritis, including rheumatoid arthritis, degenerative joint diseases (osteoarthritis), gout and ankylosing spondylitis, acute, subacute and chronic musculoskeletal pain syndromes such as bursitis, burns, injuries, and pain following surgical and dental procedures as well as the preemptive treatment of surgical pain. In addition, the combination of this invention may inhibit cellular neoplastic transformations and metastic tumor growth and hence can be used in the treatment of cancer. The combination of this invention may also be useful for the treatment or prevention of endometriosis and Parkinson's disease.

The combination of this invention will also inhibit prostanoid-induced smooth muscle contraction by preventing the synthesis of contractile prostanoids and hence may be of use in the treatment of dysmenorrhea, premature labor and asthma.

The present invention will prove useful as an alternative to conventional non-steroidal antiinflammatory drugs (NSAID'S) particularly where such non-steroidal antiinflammatory drugs may be contra-indicated such as in patients with peptic ulcers, gastritis, regional enteritis, ulcerative colitis, diverticulitis or with a recurrent history of gastrointestinal lesions; GI bleeding, coagulation disorders including anemia such as hypoprothrombinemia, haemophilia or other bleeding problems (including those relating to reduced or impaired platelet function); kidney disease (e.g. impaired renal function); those prior to surgery or taking anticoagulants; and those susceptible to NSAID induced asthma.

Similarly, the present invention will be useful as a partial or complete substitute for conventional NSAIDs in preparations wherein they are presently co-administered with other agents or ingredients. Thus in further aspects, the invention encompasses pharmaceutical compositions for treating cyclooxygenase-2 mediated diseases as defined above comprising a pharmaceutical composition comprising a cyclooxygenase-2 selective inhibitor and compound of Formula I as defined above and one or more ingredients such as another pain reliever including acetominophen or phenacetin; opioid analgesics, such as codeine, fentanyl, hydromorphone, levorphanol, meperidine, methadone, morphine, oxycodone, oxymorphine, propoxyphene, buprenorphine, butorphanol, dezocine, nalbuphine and pentazocine; a potentiator including caffeine; an H2-antagonist; aluminum or magnesium hydroxide; simethicone; a decongestant including phenylephrine, phenylpropanolamine, pseudophedrine, oxymetazoline, ephinephrine, naphazoline, xylometazoline, propylhexedrine, or levo-desoxyephedrine; an antitussive including codeine, hydrocodone, caramiphen, carbetapentane, or dextramethorphan; a diuretic; a sedating or non-sedating antihistamine; and a proton pump inhibitor, such as omeprazole. For the treatment or prevention of migraine, the invention also encompasses co-administration with a 5-HT agonist such as rizatriptan, sumatriptan, zolmitriptan and naratriptan. In addition the invention encompasses a method of treating cyclooxygenase mediated diseases comprising: administration to a patient in need of such treatment a non-toxic therapeutically effect amount of the compound of Formula I, optionally co-administered with one or more of such ingredients as listed immediately above.

The active agents of the combination of the inventions may be administered orally, topically, parenterally, by inhalation spray or rectally in dosage unit formulations containing conventional non-toxic pharmaceutically acceptable carriers, adjuvants and vehicles. The term parenteral as used herein includes subcutaneous injections, intravenous, intramuscular, intrasternal injection or infusion techniques. In addition to the treatment of warm-blooded animals such as mice, rats, horses, cattle sheep, dogs, cats, etc., the compound of the invention is effective in the treatment of humans.

As indicated above, pharmaceutical compositions for treating cyclooxygenase-2 mediated diseases as defined may optionally include one or more ingredients as listed above.

The pharmaceutical compositions containing the active ingredients may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example, magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the technique described in the U.S. Pat. Nos. 4,256,108; 4,166,452; and 4,265,874 to form osmotic therapeutic tablets for control release.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredients is mixed with water or an oil medium, for example peanut oil, liquid paraffin, or olive oil.

Aqueous suspensions contain the active material in admixture with excipients suitable for the manufacture of aqueous suspensions. Such excipients are suspending agents, for example sodium carboxymethyl-cellulose, methyl-cellulose, hydroxypropylmethyl-cellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose, saccharin or aspartame.

Liquid formulations include the use of self-emulsifying drug delivery systems and NanoCrystal® technology. Cyclodextrin inclusion complexes can also be utilized.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the invention may also be in the form of an oil-in-water emulsions. The oily phase may be a vegetable oil, for example olive oil or arachis oil, or a mineral oil, for example liquid paraffin or mixtures of these. Suitable emulsifying agents may be naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavouring agents.

Syrups and elixirs may be formulated with sweetening agents, for example glycerol, propylene glycol, sorbitol or sucrose. Such formulations may also contain a demulcent, a preservative and flavoring and coloring agents. The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The active agents of the present invention may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

For topical use, creams, ointments, jellies, solutions or suspensions, etc., containing the active agents are employed. (For purposes of this application, topical application shall include mouth washes and gargles.)

Pharmaceutical compositions of the invention may also utilize absorption enhancers such as tween 80, tween 20, Vitamin E TPGS (d-alpha-tocopheryl polyethylene glycol 1000 succinate) and Gelucire®.

Dosage levels of cycloxygenase-2 selective inhibitors are known in the art. Typically, such dosage levels may be on the order of from about 0.01 mg to about 140 mg/kg of body weight per day are useful in the treatment of the above-indicated conditions, or alternatively about 0.5 mg to about 7 g per patient per day. For example, inflammation may be effectively treated by the administration of from about 0.01 to 50 mg of the compound per kilogram of body weight per day, or alternatively about 0.5 mg to about 3.5 g per patient per day, preferably 2.5 mg to 1 g per patient per day.

The amount of active ingredients that may be combined with the carrier materials to produce a single dosage form will vary depending upon the host treated and the particular mode of administration. For example, a formulation intended for the oral administration of humans may contain from 0.5 mg to 5 g of each active agent compounded with an appropriate and convenient amount of carrier material which may vary from about 5 to about 95 percent of the total composition. Dosage unit forms will generally contain between from about 1 mg to about 500 mg of an active ingredient, typically 25 mg, 50 mg, 100 mg, 200 mg, 300 mg, 400 mg, 500 mg, 600 mg, 800 mg, or 1000 mg.

It will be understood, however, that the specific dose level for any particular patient will depend upon a variety of factors including the age, body weight, general health, sex, diet, time of administration, route of administration, rate of excretion, drug combination and the severity of the particular disease undergoing therapy.

Compounds of Formula I may contain one or more asymmetric centers and can thus occur as racemates and racemic mixtures, single enantiomers, diastereomeric mixtures and individual diastereomers. The present invention is meant to comprehend all such isomeric forms of the compounds of Formula I.

Some of the compounds described herein contain olefinic double bonds, and unless specified otherwise, are meant to include both E and Z geometric isomers.

Some of the compounds described herein may exist with different points of attachment of hydrogen, referred to as tautomers. Such an example may be a ketone and its enol form known as keto-enol tautomers. The individual tautomers as well as mixture thereof are encompassed with compounds of formula I.

Compounds of the Formula I may be separated into diastereoisomeric pairs of enantiomers by, for example, fractional crystallization from a suitable solvent, for example methanol or ethyl acetate or a mixture thereof. The pair of enantiomers thus obtained may be separated into individual stereoisomers by conventional means, for example by the use of an optically active acid as a resolving agent.

Alternatively, any enantiomer of a compound of the general formula I may be obtained by stereospecific synthesis using optically pure starting materials or reagents of known configuration.

The invention also includes the compounds falling within Formula I in the form of one or more stereoisomers, in substantially pure form or in the form of a mixture of stereoisomers. All such isomers are encompassed within the present invention.

### Assays for Determining Biological Activity

The gastric protective effects of the combination of the present invention co-administered with aspirin may be evaluated in the following assay.

Male Wistar rats (200-250 g) were fasted for 16-18 h prior to use for experiment. Aspririn, rofecoxib in combination with aspirin (dosed separately), or test compound in combination with rofecoxib and aspirin (dosed separately) were given on the morning of the experiment at a dosing volume of 1 ml/kg in 0.5% methocel. Three hr later, the animals were euthanized by CO₂ inhalation and the stomach removed, rinsed in saline and prepared for imaging processing. Microscopic pictures of the stomach were taken using a digital camera and gastric erosions were measured using an imaging software by an observer unaware of the treatment groups. The length of gastric erosions was measured in mm and the total length of all erosions from each stomach was obtained and used as gastric damage score.

This model is also described in S. Fiorucci, et al., Gastroenterology, vol. 123, pp. 1598-1606, 2002 and M. Souza, et al., Am. J. Physiol. Gastrointest. Liver Physiol., vol. 285, pp. G54-G61, 2003.

### Methods of Synthesis

Compounds of the invention may be synthesized by according to the following synthetic scheme:

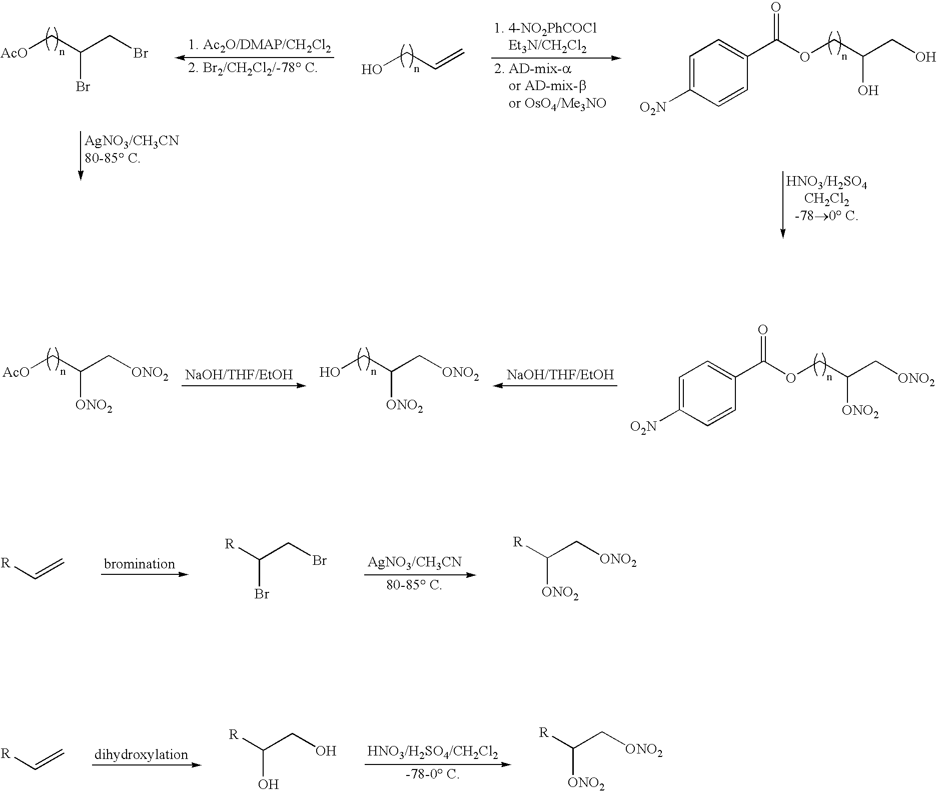

### REPRESENTATIVE EXAMPLES

Compounds of Formula I are exemplified as follows:

### Example 1

### 5,6-bis(nitrooxy)hexyl acetate

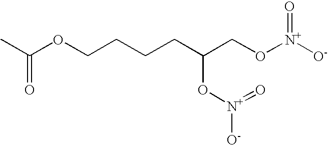

### Step 1: 5,6-dibromohexyl acetate

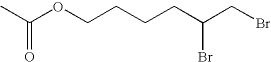

To a solution of hex-5-en-1-ol (30.9 g, 309 mmol) and DMAP (41.5 g, 340 mmol, 1.1 equiv) in CH₂Cl₂ (750 mL, 0.4 M) at −78° C., was added acetic anhydride (32.1 mL, 340 mmol, 1.1 equiv). The reaction was stirred at rt for 1 h. The product was passed through a plug of silica gel using 20% EA/Hex to afford the desired product (42.7 g, Yield=97%) as a colorless oil. The compound was used directly for bromination. To a solution of hex-5-en-lyl acetate (42.7 g, 300 mmol) in CH₂Cl₂ (600 mL, 0.5 M) at −78° C., was added bromine (330 mL, 330 mmol, 1.1 equiv). The reaction was stirred at −78° C. for 15 min then warmed to rt. The solvent was evaporated to afford the desired product (89 g, Yield=98%) as a yellowish oil. ¹H NMR (500 MHz, Acetone): δ 4.38-4.32 (m, 1H), 4.04 (t, 2H), 3.95-3.93 (m, 1H), 3.84-3.80 (m, 1H), 2.16-2.10 (m, 1H), 1.98 (s, 3H), 1.90-1.82 (m, 1H), 1.72-1.62 (m, 3H), 1.56-1.48 (m, 1H).

### Step 2: 5,6-bis(nitrooxy)hexyl acetate

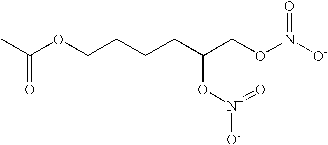

To a solution of 5,6-dibromohexyl acetate (89 g, 2951 mmol) in CH₃CN (600 mL, 0.282 M) at rt was added silver nitrate (200 g, 1180 mmol, 4 equiv). The reaction was stirred at 80° C. overnight then evaporated to dryness. The residue was suspended in EtOAc, sonicated and filtered through a plug of silica gel and washed with EA. The product was purified by combi-flash 3×120 g silica gel cartridge using gradient (0-5% in 5 min, 5-55% in 30 min, 55-70% EA/Hex in 10 min) to afford the desired product (65.2 g, Yield=83%) as a yellowish oil. ¹H NMR (500 MHz, Acetone): δ 5.52-5.46 (m, 1H), 5.00 (dd, 1H), 4.72 (dd, 1H), 4.05-4.03 (m, 2H), 1.96 (s, 3H), 1.88-1.84 (m, 2H), 1.71-1.65 (m, 2H), 1.60-1.50 (m, 2H).

### Example 2

### 6-hydroxyhexane-1,2-diyl dinitrate

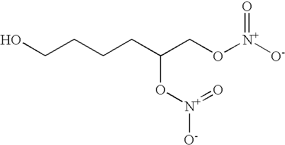

To a solution of 5,6-bis(nitrooxy)hexyl acetate (31.3 g, 118 mmol) in THF-EtOH (1:1, 0.492 M) at 0° C., was added a solution of sodium hydroxide (2 N, 126 mL, 251 mmol, 2.1 equiv) dropwise over 5 min. The reaction was stirred at rt for 2 h. The reaction mixture was quenched with a saturated NaHCO₃ solution and extracted 3 times with EA. The combined organic layers were washed with brine, dried over Na₂SO₄, filtered and evaporated. The product was purified by combi-flash 2×120 g silica gel cartridge using gradient (0-5% in 5 min, 5-55% in 30 min, 55-70% EA/Hex in 10 min) to afford the desired product (24.5 g, Yield=92%) as a pale yellowish oil. ¹H NMR (500 MHz, Acetone): δ 5.53-5.47 (m, 1H), 5.00 (dd, 1H), 4.72 (dd, 1H), 3.55 (d, 2H), 3.50 (t, 1H), 1.88-1.80 (m, 2H), 1.58-1.51 (m, 4H).

### Example 3

### 5-hydroxypentane-1,2-diyl dinitrate

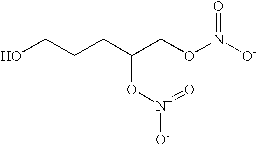

To a solution of pent-4-en-1-yl acetate (30 g, 234 mmol) in dichloromethane (468 mL, 0.5 M) at −78° C., was added bromine (257 mL, 257 mmol, 1.1 equiv). The reaction was stirred at −78° C. for 15 min then warm-up to rt. The reaction mixture was then evaporated to afford the desired compound (67 g, Yield=99%) which was used directly for the next step. To a solution of 4,5-dibromopentyl acetate (75 g, 260 mmol) in acetonitrile (600 mL, 0.433 M) at rt, was added silver nitrate (177 g, 1040 mmol, 4 equiv). The reaction was stirred at 80° C. overnight. The solvent was removed by evaporation, the crude reaction mixture was suspended in EtOAc, filtered through a pad of silica gel, washed with EtOAc (1 L) then evaporated. The desired compound (65.5 g, Yield=99%) was obtained as a yellowish oil and used directly for the next step. To a solution of 4,5-bis(nitrooxy)pentyl acetate (26 g, 103 mmol) in THF-EtOH (1:1, 0.468 M) at 0° C., was added a solution of sodium hydroxide (2 N, 110 mL, 219 mmol, 2.1 equiv) dropwise over 5 min. The reaction was stirred at rt for 2 h. The reaction mixture was quenched with a saturated NaHCO₃ solution, diluted and extracted 3 times with EtOAc. The combined organic layers were washed with brine, dried over Na₂SO₄, filtered and concentrated. The product was purified by combi-flash 120 g silica gel cartridge using gradient (0-5% in 5 min, 5-55% in 30 min, 55-70% EA/Hex in 10 min) to afford the desired product (11.5 g, Yield=53%) as a pale yellowish oil. ¹H NMR (500 MHz, Acetone): δ 5.60-5.56 (m, 1H), 5.06-4.98 (m, 1H), 4.76 (dd, 1H), 3.80 (t, 1H), 3.63 (m, 2H), 2.00-1.86 (m, 2H), 1.73-1.67 (m, 2H).

### Example 4

### (5R)-5,6-bis(nitrooxy)hexyl 4-nitrobenzoate

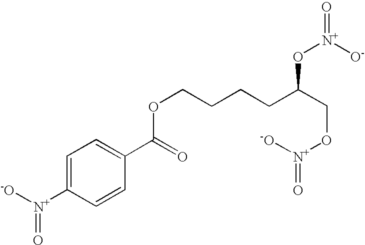

### Step 1: hex-5-en-1-yl 4-nitrobenzoate

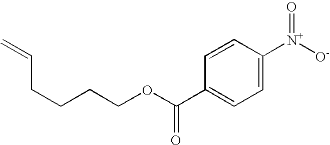

To a solution of hex-5-en-1-ol (20.0 g, 200 mmol) in CH₂Cl₂ (350 mL, 0.57 M) at 0° C. was added triethylamine (33.7 mL, 240 mmol, 1.2 equiv), DMAP (1.22 g, 10 mmol, 0.05 equiv) and then 4-nitrobenzoyl chloride (39 g, 210 mmol, 1.05 equiv.). The reaction was stirred at rt for 2 h. The reaction mixture was quenched with a saturated NH₄Cl solution and extracted 3 times with CH₂Cl₂. The combined organic layers were washed with brine, dried over Na₂SO₄, filtered and evaporated. The product was purified by combi-flash 2×120 g silica gel cartridge using gradient (0-5% in 5 min, 5-25% in 30 min, 25-40% EA/Hex in 10 min) to afford the desired product (35 g, Yield=70%) as a yellowish oil. ¹H NMR (500 MHz, Acetone): δ 8.36 (d, 2H), 8.26 (d, 2H), 5.87-5.79 (m, 1H), 5.03 (d, 1H), 4.94 (d, 1H), 4.38 (t, 2H), 2.14 (m, 2H), 1.84-1.78 (m, 2H), 1.60-1.54 (m, 2H).

### Step 2: (5R)-5,6-dihydroxyhexyl 4-nitrobenzoate

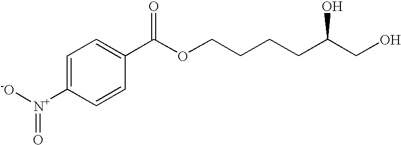

To a solution of AD-mix-beta (34 g) in t-BuOH-H₂O (1:1, 0.077 M) at 0° C., was added hex-5-en-1-yl 4-nitrobenzoate (6 g, 24.07 mmol). The reaction was stirred at 0° C. overnight. The reaction mixture was diluted with 250 mL of EA, quenched by addition of 10 g of sodium metabisulfite and stirred 30 min at 0° C. The mixture was allowed to stir at rt for 1 h then was extracted 3 times with EtOAc, washed with brine, dried over Na₂SO₄, filtered and evaporated. The compound was recrystallized with ether and stirred in the cold room overnight for twice to afford 3.3 g of the titled compound as a white solid (greater than 99% in e.e. by Mosher derivatives analysis). ¹H NMR (500 MHz, Acetone): δ 8.36 (d, 2H), 8.27 (d, 2H), 4.38 (t, 2H), 3.62-3.54 (m, 3H), 3.49-3.45 (m, 1H), 3.41-3.35 (m, 1H), 1.87-1.77 (m, 2H), 1.69-1.62 (m, 1H), 1.59-1.49 (m, 2H), 1.46-1.40 (m, 1H).

### Step 3: (5R)-5,6-bis(nitrooxy)hexyl 4-nitrobenzoate

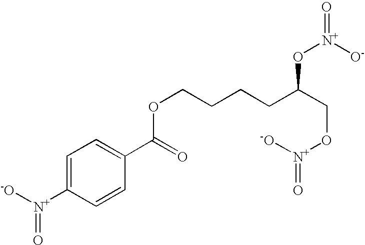

To a solution of nitric acid (8 mL, 178 mmol, 15.75 equiv) in CH₂Cl₂ (20 mL, 0.565 M) at −78° C., was added sulfuric acid (2 mL, 37.5 mmol, 3.32 equiv) and then a solution of (5R)-5,6-dihydroxyhexyl 4-nitrobenzoate (2.5 g, 8.83 mmol) in CH₂Cl₂ (10 mL). The reaction was stirred at 0° C. for 2 h. The reaction mixture was poured on ice (ca 200 g) and extracted 3 times with CH₂Cl₂. The combined organic layers were washed with water, brine, dried over Na₂SO₄, filtered and evaporated. The product was purified by combi-flash 120 g silica gel cartridge using gradient (0-5% in 5 min, 5-40% in 25 min, 40-70% EA/Hex in 10 min) to afford the desired product (3.1 g, Yield=91%) as a viscous yellowish oil. ¹H NMR (500 MHz, Acetone): δ 8.35 (d, 2H), 8.26 (d, 2H), 5.56-5.52 (m, 1H), 5.03 (dd, 1H), 4.75 (dd, 1H), 4.41 (t, 2H), 1.98-1.88 (m, 4H), 1.76-1.66 (m, 2H).

### Example 5

### (5S)-5,6-bis(nitrooxy)hexyl 4-nitrobenzoate

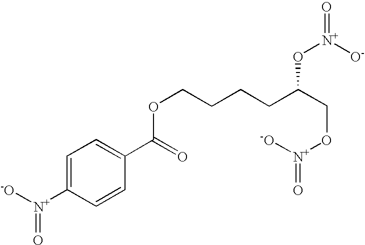

The titled compound was prepared by following the procedures described in Step 1-3 of EXAMPLE 4 except that the reagent AD-mix-beta used in Step 2 was replaced with AD-mix-alpha.

### Example 6

### (2R)-6-hydroxyhexane-1,2-diyl dinitrate

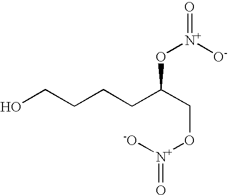

To a solution of (5R)-5,6-bis(nitrooxy)hexyl 4-nitrobenzoate (3.1 g, 8.3 mmol) in THF-EtOH (1:1, 0.5 M) at 0° C. was added sodium hydroxide 2N (10 mL, 20 mmol, 2 equiv) dropwise over 5 min. The reaction was stirred at rt for 2 h. The reaction mixture was quenched with a saturated NaHCO₃ solution and extracted 3 times with EtOAc. The combined organic layers were washed with brine, dried over Na₂SO₄, filtered and evaporated. The product was purified by combi-flash 120 g silica gel cartridge using gradient (0-5% in 5 min, 5-55% in 30 min, 55-70% EA/Hex in 10 min) to afford the desired product (1.51 g, Yield=81%) as a colorless oil. ¹H NMR (500 MHz, Acetone): δ 5.52-5.48 (m, 1H), 5.01 (dd, 1H), 4.72 (dd, 1H), 3.56-3.50 (m, 3H), 1.88-1.81 (m, 2H), 1.54-1.50 (m, 4H).

### Example 7

### (2S)-6-hydroxyhexane-1,2-diyl dinitrate

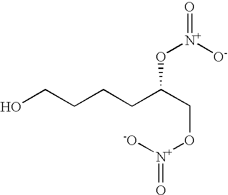

The titled compound was prepared by following the procedures in EXAMPLE 6 except that the reagent (5R)-5,6-bis(nitrooxy)hexyl 4-nitrobenzoate used was replaced with (5S)-5,6-bis(nitrooxy)hexyl 4-nitrobenzoate.

### Example 8

### (2S)-propane-1,2-diyl dinitrate

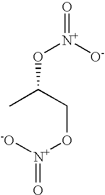

To a solution of nitric acid (200 mL) in CH₂Cl₂ (400 mL) at −78° C., was added sulfuric acid (50 mL) and then a solution of (2S)-propane-1,2-diol (10 g) in CH₂Cl₂ (50 mL). The reaction was stirred at 0° C. for 3 h. The reaction mixture was poured on ice (ca 1000 g) and extracted 3 times with CH₂Cl₂. The combined organic layers were washed with water, brine, dried over Na₂SO₄, filtered and evaporated to afford the desired product (18.6 g) as yellowish oil. ¹H NMR (500 MHz, Acetone): δ 5.62-5.53 (m, 1H), 4.94 (dd, 1H), 4.71 (dd, 1H), 1.46 (d, 3H).

### Example 9

### (2R)-propane-1,2-diyl dinitrate

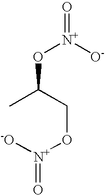

The titled compound was prepared by following the procedures in EXAMPLE 8 except that the reagent (2S)-propane-1,2-diol used was replaced with (2R)-propane-1,2-diol.

Additional examples of the invention include compounds of Formula II or compounds of Formula III:

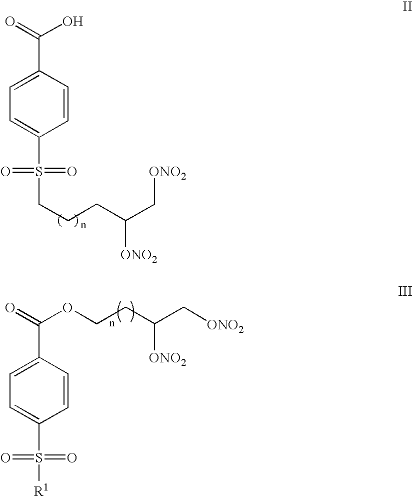

wherein R¹ is C₁₋₆alkyl and n is an integer from 1 to 8.

1. A method for treating a cyclooxygenase-2 mediated disease or condition in a human patient at risk of a thrombotic cardiovascular event, wherein the patient is on aspirin therapy to reduce the risk of the thrombotic cardiovascular event,
comprising orally concomitantly or sequentially administering to the patient a cyclooxygenase-2 selective inhibitor in an amount effective to treat the cyclooxygenase-2 mediate disease or condition, and
a nitric oxide donating compound in accordance with Formula I 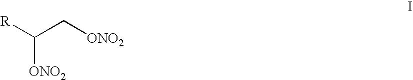
or a pharmaceutically acceptable salt thereof, wherein R is selected from the group consisting of: 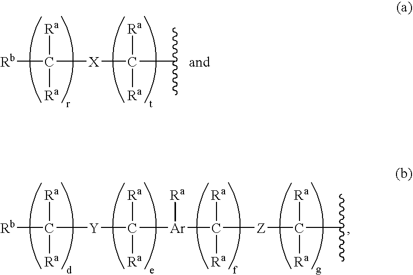
wherein:
r and t are independently 0 to 10,
d, e, f and g are independently 0 to 10;
Ar is selected from the group consisting of: phenyl, naphthyl, biphenyl and HET,
X, Y and Z are independently selected from the group consisting of: a bond, —O—, —S(O)ₖ—, —O—C(O)—, —C(O)—O— and —O—C(O)—O—, with the proviso that when X is other than a bond then r is not 0, and with the proviso that when Y is other than a bond then d is not 0, and wherein k is 0, 1 or 2,
each R^{a} and R^{b} are independently selected from the group consisting of:
(1) hydrogen,
(2) halo,
(3) C₁₋₄alkoxy,
(4) C₁₋₄alkylthio,
(5) OH,
(6) CN,
(7) CO₂R^{c},
(8) —C₀₋₆—ONO₂,
and two R^{a} groups on the same carbon atom may be joined together to form a carbonyl group;
R^{c} is selected from the group consisting of: hydrogen and C₁₋₆alkyl; and
HET is selected from the group consisting of: benzimidazolyl, benzofuranyl, benzopyrazolyl, benzotriazolyl, benzothiophenyl, benzoxazolyl, carbazolyl, carbolinyl, cinnolinyl, furanyl, imidazolyl, indolinyl, indolyl, indolazinyl, indazolyl, isobenzofuranyl, isoindolyl, isoquinolyl, isothiazolyl, isoxazolyl, naphthyridinyl, oxadiazolyl, oxazolyl, pyrazinyl, pyrazolyl, pyridopyridinyl, pyridazinyl, pyridyl, pyrimidyl, pyrrolyl, quinazolinyl, quinolyl, quinoxalinyl, thiadiazolyl, thiazolyl, thienyl, triazolyl, azetidinyl, 1,4-dioxanyl, hexahydroazepinyl, piperazinyl, piperidinyl, pyrrolidinyl, morpholinyl, thiomorpholinyl, dihydrobenzimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, and tetrahydrothienyl,
wherein the nitric oxide donating compound is administered in an amount effective to reduce the gastrointestinal toxicity caused by the combination of the cyclooxygenase-2 selective inhibitor and aspirin.

2-3. (canceled)

4. The method according to claim 1 wherein the cyclooxygenase-2 selective inhibitor is selected from the group consisting of: rofecoxib, etoricoxib, celecoxib, valdecoxib, lumiracoxib, LAS34475 and GW406381.

5. The method according to claim 4 wherein the cyclooxygenase-2 selective inhibitor is rofecoxib.

6. The method according to claim 1 wherein R is 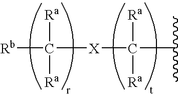
wherein X is a bond, each R^{a} is hydrogen and R^{b} is OH.

7. The compound according to claim 1 wherein R is 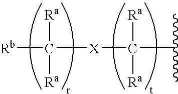
wherein X is a —O—C(O)— or —C(O)—O—, each R^{a} is hydrogen and R^{b} is hydrogen and both r and t are greater than 0.

8. The method according to claim 1 wherein R is 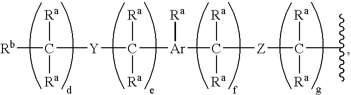
wherein Ar is phenyl,
f and e are both 0 and
Z and Y are independently S(O)₂, —O—C(O)— or —C(O)—O—.

9. The method according to claim 1 wherein the nitric oxide donating compound in accordance with Formula I is selected from the group consisting of:

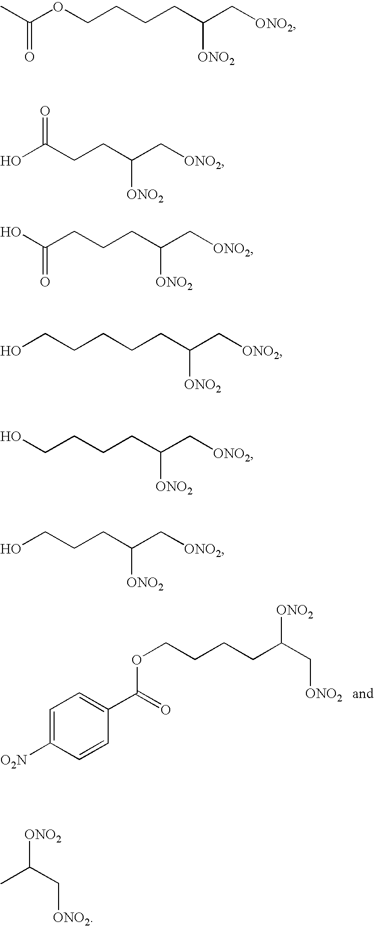

10. The method according to claim 1 wherein R is selected from 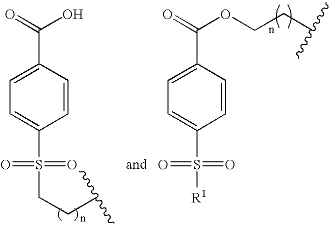
wherein R¹ is C₁₋₆alkyl and n is 1 to 8.

11. The method according to claim 1 wherein the thrombotic cardiovascular event is selected from the group consisting of: thrombotic or thromboembolic stroke, myocardial ischemia, myocardial infarction, angina pectoris, transient ischemic attack and reversible ischemic neurologic deficits.

12. The method according to claim 1 wherein the cyclooxygenase-2 mediated disease or condition is selected from the group consisting of: osteoarthritis, rheumatoid arthritis, chronic and acute pain, primary dysmenorrhea, gout, ankylosing spondylitis and bursitis.

13. A pharmaceutical composition comprising a nitric oxide donating compound in accordance with Formula I 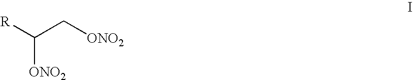
or a pharmaceutically acceptable salt thereof, wherein R is selected from the group consisting of: 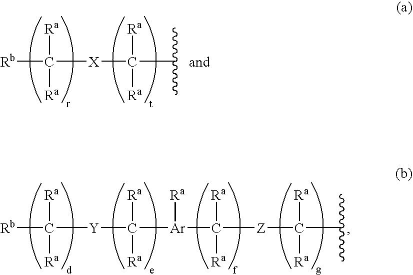
wherein:
r and t are independently 0 to 10,
d, e, f and g are independently 0 to 10;
Ar is selected from the group consisting of: phenyl, naphthyl, biphenyl and HET,
X, Y and Z are independently selected from the group consisting of: a bond, —O—, —S(O)ₖ—, —O—C(O)—, —C(O)—O— and —O—C(O)—O—, with the proviso that when X is other than a bond then r is not 0, and with the proviso that when Y is other than a bond then d is not 0, and wherein k is 0, 1 or 2,
each R^{a} and R^{b} are independently selected from the group consisting of:
(1) hydrogen,
(2) halo,
(3) C₁₋₄alkoxy,
(4) C₁₋₄alkylthio,
(5) OH,
(6) CN,
(7) CO₂R^{c} and
(8) —C₀₋₆—ONO₂,
R^{c} is selected from the group consisting of: hydrogen and C₁₋₆alkyl; and
HET is selected from the group consisting of: benzimidazolyl, benzofuranyl, benzopyrazolyl, benzotriazolyl, benzothiophenyl, benzoxazolyl, carbazolyl, carbolinyl, cinnolinyl, furanyl, imidazolyl, indolinyl, indolyl, indolazinyl, indazolyl, isobenzofuranyl, isoindolyl, isoquinolyl, isothiazolyl, isoxazolyl, naphthyridinyl, oxadiazolyl, oxazolyl, pyrazinyl, pyrazolyl, pyridopyridinyl, pyridazinyl, pyridyl pyrimidyl, pyrrolyl, quinazolinyl, quinolyl, quinoxalinyl, thiadiazolyl, thiazolyl, thienyl, triazolyl, azetidinyl, 1,4-dioxanyl, hexahydroazepinyl, piperazinyl, piperidinyl, pyrrolidinyl, morpholinyl, thiomorpholinyl, dihydrobenzimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, and tetrahydrothienyl,
and a cyclooxygenase-2 selective inhibitor in combination with a pharmaceutically acceptable carrier.

14. The pharmaceutical composition according to claim 13 wherein the cyclooxygenase-2 selective inhibitor is selected from the group consisting of: rofecoxib, etoricoxib, celecoxib, valdecoxib, lumiracoxib, LAS34475 and GW406381.

15. The pharmaceutical composition according to claim 14 wherein the cyclooxygenase-2 selective inhibitor is rofecoxib.

16. The pharmaceutical composition according to claim 15 wherein rofecoxib is present in amount selected from the group consisting of 12.5 mg, 25 mg and 50 mg.

17. The pharmaceutical composition according to claim 16 comprising rofecoxib present in an amount selected from the group consisting of 12.5 mg, 25 mg and 50 mg and a nitric oxide donating compound having the following formula: 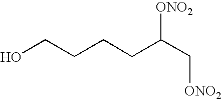
in combination with a pharmaceutically acceptable carrier.

18. The pharmaceutical composition according to claim 13 wherein R is 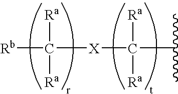
wherein X is a bond, each R^{a} is hydrogen and R^{b} is OH.

19. The pharmaceutical composition according to claim 13 wherein R is 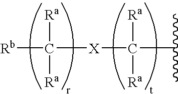
wherein X is a —O—C(O)— or —C(O)—O—, each R^{a} is hydrogen and R^{b} is hydrogen and both r and t are greater than 0.

20. The pharmaceutical composition according to claim 13 wherein R is 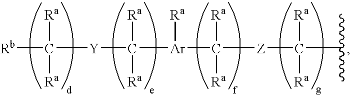
wherein Ar is phenyl,
f and e are both 0 and
Z and Y are independently S(O)₂, —O—C(O)— or —C(O)—O—.

21. The pharmaceutical composition according to claim 13 wherein the nitric oxide donating compound in accordance with Formula I is selected from the group consisting of:

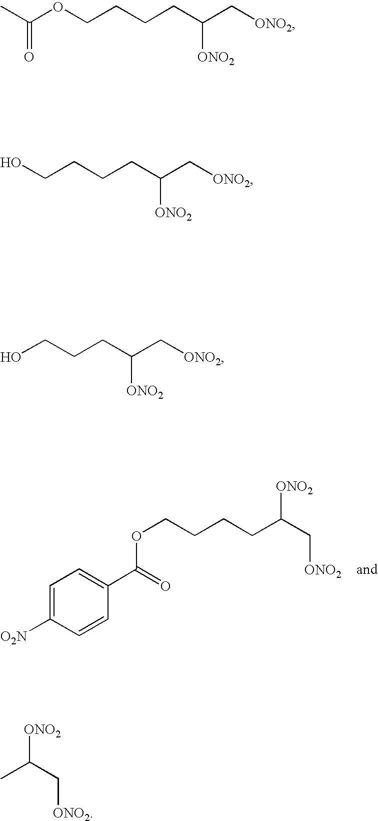

22. The pharmaceutical composition according to claim 13 wherein R is selected from 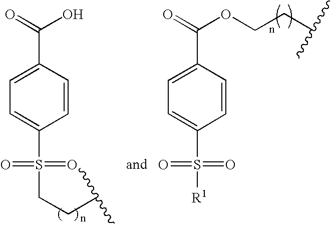
wherein R¹ is C₁₋₆alkyl and n is 1 to 8.

23. A method for treating a cyclooxygenase-2 mediated disease or condition comprising administering the pharmaceutical composition according to claim 13.

24. The method according to claim 23 wherein the cyclooxygenase-2 mediated disease or condition is selected from the group consisting of: osteoarthritis, rheumatoid arthritis, chronic and acute pain, primary dysmenorrhea, gout, ankylosing spondylitis and bursitis.

25. The method according to claim 23 wherein the patient is at risk of a thrombotic cardiovascular event and wherein the patient is on aspirin therapy to reduce the risk of the thrombotic cardiovascular event.

26-29. (canceled)
